(19) United States
(12) Patent Application Publication
(10) Pub. No.: US 2014/0056764 A1
(43) Pub. Date: Feb. 27, 2014

(54) ODOUR CONTROL UNIT

(71) Applicant: Neil Verner, Welwyn Garden City (GB)

(72) Inventor: Neil Verner, Welwyn Garden City (GB)

(73) Assignee: SIRIUS PRODUCTS LIMITED, Welwyn Garden City, Hertfordshire (GB)

(21) Appl. No.: 13/981,895

(22) PCT Filed: Jan. 25, 2012
(86) PCT No.: PCT/GB2012/000074

§ 371 Date: Nov. 12, 2013

(30) Foreign Application Priority Data
Jan. 25, 2011 (GB) 1101268.9

(51) Int. Cl. A61L 9/015

(52) U.S. Cl. CPC A61L 9/015
USPC 422/119

(57) ABSTRACT

An odour control unit comprising an input conduit, an output conduit, a means for generating ultra-violet light which in turn can generate ozone and a means for measuring the concentration of ozone present in the output conduit, wherein the rate of ozone generation can be varied depending on the concentration of ozone in the output conduit.

The present invention relates to an odour removal unit, especially but not exclusively for use in the catering industry.

Odorous exhaust fumes are well known unwanted by-products of the process of cooking food. Kitchens that operate on a large scale, such as in restaurants or canteens can generate a large volume of exhaust fumes. This can present a major problem when the kitchen is located in a densely-populated area. Traditionally, kitchens have been equipped with extractor fans which may direct the exhaust fumes into a chimney so as to remove them away from people likely to be affected by them. More recently extractor fans have been fitted with filters which aim to remove from the fumes particulate matter above a certain particle size.

Unfortunately, some of the odour-causing chemicals in a vaporised state within such fumes are too small to be caught by a straightforward filter. The result is that filtered exhaust fumes may still be significantly odorous when released into the environment.

It is an aim of the present invention to reduce the emission of odorous kitchen exhaust fumes into the environment.

Accordingly, a first aspect of the present invention is directed to an odour control unit comprising an input conduit, an output conduit, a means for generating ultra-violet light which in turn can generate ozone and a means for measuring the concentration of ozone present in the output conduit, wherein the rate of ozone generation can be varied depending on the concentration of ozone in the output conduit.

Ozone may be generated by the interaction of oxygen in the air and ultra-violet light. An effective wavelength of ultra-violet light for ozone generation is approximately 185 μm.

Ozone is highly reactive and can be used to oxidise or otherwise denature compounds which come into contact with it. It can be generated in situ by the action of ultra-violet light on ambient oxygen-bearing air.

However due to ozone's high reactivity, it is generally regarded as undesirable to allow its concentration to build up in the environment. Therefore the ability to generate enough ozone to neutralise odours in the exhaust fumes without producing an undesirable excess is significantly advantageous.

The rate of ozone generation may be varied to prevent the concentration of ozone in the output conduit from exceeding a preset value.

This feedback arrangement ensures that the concentration of ozone in the output conduit does not exceed safe levels when the throughput of odorous material is low whilst also ensuring that, if the throughput of odorous material increases, the odour removal can be increased.

The unit may further comprise inner surfaces which are at least partially coated with reflective material.

A reflective inner surface maximises the odour removal effectiveness of the ultra-violet light by minimising the amount of radiation which is absorbed by the surfaces of the unit.

At least some of the inner surfaces may be corrugated.

The unit may further comprise inner surfaces which are at least partially coated with material which, when ultra-violet light of wavelength approximately 254 μm is incident upon it, enters a superhydrophilic state and acts as a catalyst for the oxidisation of certain compounds contained within the odorous fumes.

Moisture from the air passing through the unit interacts with the superhydrophilic material to allow the inner surfaces of the unit to be self-cleaning.

The superhydrophilic material may be titanium dioxide in the anatase phase.

Titanium dioxide in the anatase phase absorbs and re-emits ultra-violet radiation. In addition hydroxyl radicals are formed on the inner surface for the moisture which aid the odour removal. This process, combined with the self-cleaning performance of the inner surface maximises the odour removal efficiency of the unit.

The means for generating ultra-violet light may be one or more pellet amalgam lamp.

Pellet amalgam lamps have high efficiency at a wide range of temperatures which is advantageous in the present situation because the odorous material can vary widely in temperature and can be heated by the ultra-violet light.

There are pellet amalgam lamps available which can operate at maximum output between 25 celsius and 100 celsius.

The or each ultra-violet lamp may be protected from debris by a shield. A shield will enable the lamp to operate at optimum efficiency without its performance being degraded by becoming coated with unwanted matter from the exhaust fumes.

The unit may further comprise a filter at the input and output conduits.

The filters prevent ultra-violet radiation from escaping from the unit. Furthermore, the filters reduce the entry into and exit from the unit of particulate matter.

Each of the filters may comprise a mesh or perforated sheet.

The unit may be constructed as a box capable of receiving one or more removable cartridges. This construction enables the fitting of the unit in differing air-flow direction configurations without the need for differing versions of the unit to be available to the customer.

A second aspect of the present invention is directed to a unit comprising an exhaust air treatment unit and a bypass, wherein the flow of air passing through the exhaust air treatment unit may be temporarily diverted through the bypass.

This aspect enables the maintenance of the internal elements of the exhaust air treatment unit without interrupting extraction of exhaust air.

Known extraction systems require the entire system to be switched off during maintenance. In order to maintain adequate exhaust air extraction and fresh air supply this generally means that maintenance of an exhaust air treatment unit can only be carried out when the kitchen equipment is switched off. In the catering sector this means that maintenance can only occur at times of day which are highly anti-social. Therefore this development fulfils a long-standing need in the industry.

The exhaust air treatment unit may be an odour control unit according to the first aspect of the present invention or any suitable exhaust air treatment unit.

The flow of air through the unit may be controlled by at least one damper and at least one closing plate which determine whether the air flows through the exhaust air treatment unit or the bypass or both.

Each unit may have an inbuilt damper controlled bypass in the top section to ensure cooking can continue and the restaurant can operate during a service visit. The bypass damper may be opened allowing air to pass freely through it, then two closing plates may be fitted at the entrance and exit of the main body of the exhaust air treatment unit. When these closing plates are in place a full service can continue while the restaurant enjoys full extraction.

The bypass may be constructed integrally with or separate from the exhaust air treatment unit.

The bypass may be a second substantially similar exhaust air treatment unit.

An odour control unit made in accordance with the first aspect of the present invention will now be described hereinbelow with reference and as shown in the enclosed drawings, in which:

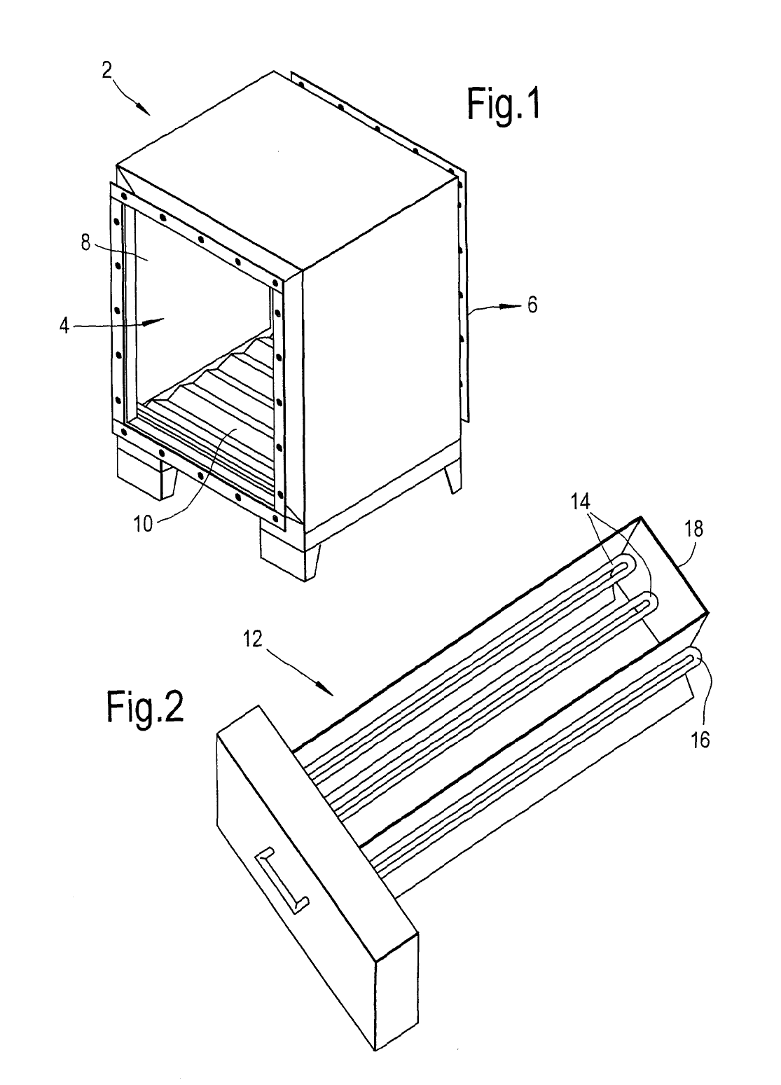

FIG. 1 shows a perspective view of a box according to the first aspect of the present invention;

FIG. 2 shows a perspective view of a cartridge capable of being received into a box to form a unit according to the first aspect of the present invention; and

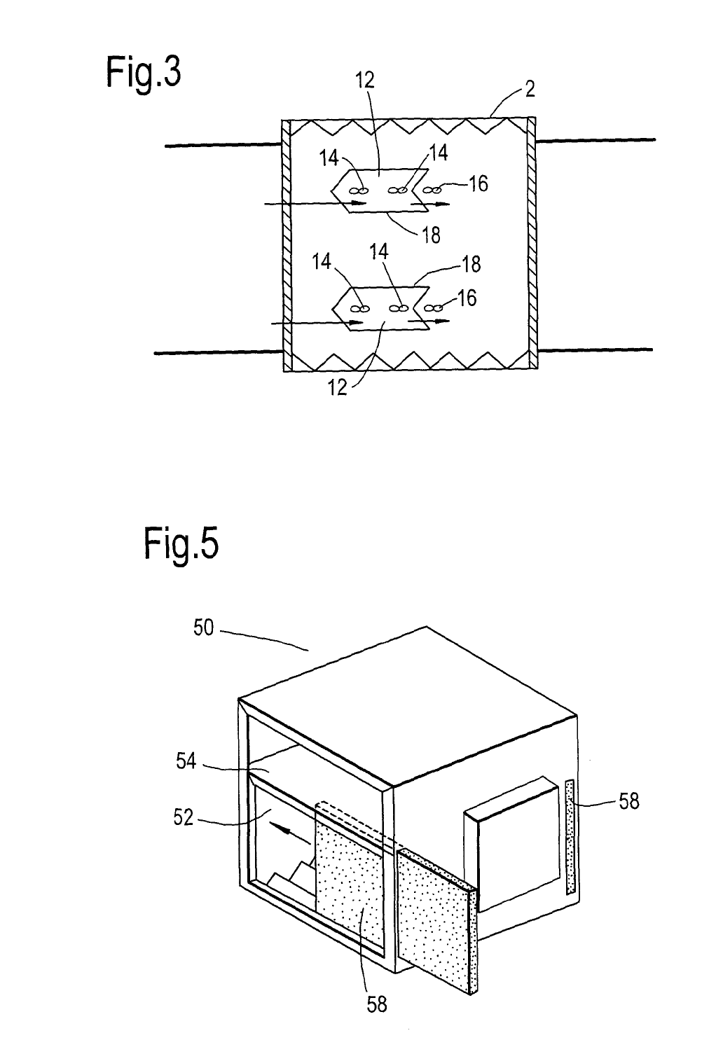

FIG. 3 shows a cross-section of a unit having a box and two cartridges received within the box according to the first aspect of the present invention.

In FIG. 1 a box 2 has an input 4 and an output 6 to allow exhaust fumes to flow through the box 2. The input 4 and output 6 are designed to be fitted to the exhaust conduit or line of a kitchen extraction system. Filters may be placed across the input and output 4, 6 in order to reduce the flow of particulate material into the box 2. An inner surface 8 of the box 2 is coated with reflective material. Another inner surface 10 of the box 2 is corrugated. One or more pellet amalgam lamps (not shown) generate ozone within the box 2. The output 6 further comprises an ozone concentration monitoring means (not shown).

FIG. 2 shows a cartridge 12 with two bulbs 14 which generate 185 μm ultra-violet light and one bulb 16 which generates 254 μm ultra-violet light. A shield 18 protects the bulbs 14, 16 from debris which may damage the bulbs 14, 16. Some or all of the surfaces within the cartridge are coated with anatase titanium dioxide.

FIG. 3 shows a unit according to the present invention comprising a box 2 within which have been received two cartridges 12.

Air is drawn by an external means (not shown) into the unit through the input 4. Within the box 2 there is generated ultra-violet light and consequently ozone gas. The ozone gas oxidises or otherwise denatures odorous chemicals. The air continues to flow from the input 4 towards the output 6. At the output 6 there is located an ozone concentration monitoring means. The measurement of ozone concentration is used to determine the required intensity of ultra-violet light generation and, as a direct consequence, the concentration of ozone generated within the box 2. This process ensures that the concentration of ozone passing out of the box 2 via the output 6 into the environment is limited to a preset level.

The unit of this particular embodiment can process approximately 1.2 m³s⁻¹ of exhaust fumes. Units according to this invention may be combined to operate in series or in parallel in order to increase the effectiveness of the odour removal or to enable a higher throughput of exhaust fumes.

A desirable maximum level of ozone at the output conduit 6 is approximately 0.06 parts per million of ozone.

Numerous variations and modifications to the illustrated odour removal unit may occur to the reader without taking the resulting construction outside the scope of the present invention.

A unit made in accordance with the second aspect of the present invention will now be described hereinbelow with reference and as shown in the enclosed drawings, in which:

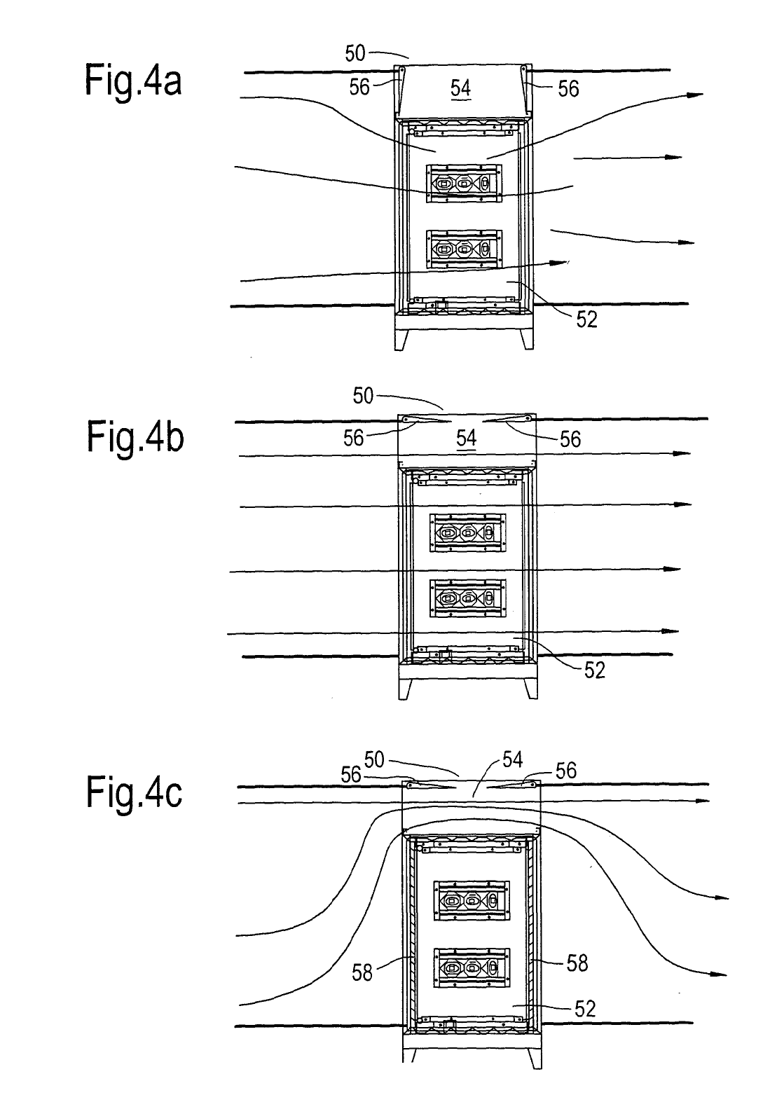

FIGS. 4a, 4b and 4c show a cross-section of a unit according to the second aspect of the present invention;

FIG. 5 shows a perspective view of a unit according to the second aspect of the present invention;

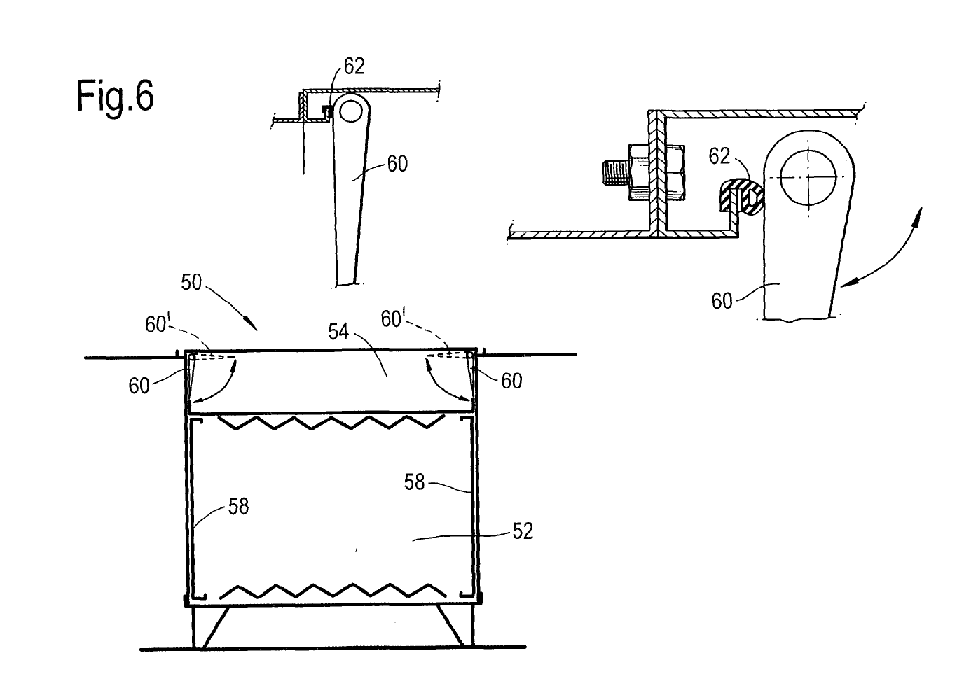

FIG. 6 shows a cross-section of a unit according to the second aspect of the present invention; and

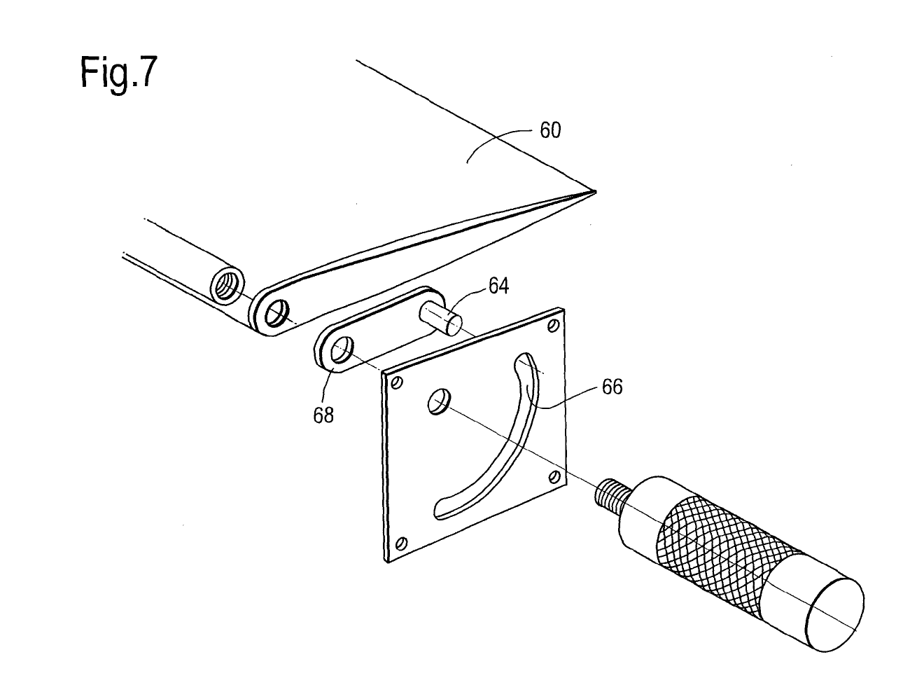

FIG. 7 shows an exploded view of a damper to be used with the second aspect of the present invention.

FIG. 4a shows the unit 50 under standard operating conditions whereby dampers 56 at either end of the bypass and closing plates (not shown) at either end of the exhaust air treatment unit 52 are set to allow air to flow only through the exhaust air treatment unit 52.

FIG. 4b shows the unit 50 in a state whereby dampers 56 at either end of the bypass 54 and closing plates (not shown) at either end of the exhaust air treatment unit 52 are set to allow air to flow through the exhaust air treatment unit 52 and the bypass 54.

FIG. 4c shows the unit 50 in a state whereby dampers 56 at either end of the bypass 54 and closing plates 58 at either end of the exhaust air treatment unit 52 are set to allow air to flow only through the bypass 54.

FIG. 5 shows a unit 50 with an exhaust air treatment unit 52 and a bypass 54 where dampers (not shown) control air flow to the bypass 54 and slidable doors 58 control air flow to the exhaust air treatment unit 52.

FIG. 6 shows a unit 50 with an exhaust air treatment unit 52 and a bypass 54 where dampers 60 control air flow to the bypass 54 and slidable doors 58 control air flow to the exhaust air treatment unit 52. An air tight seal ensures that when the dampers 60 are closed no air passes through the bypass 54. When the dampers 60 are open 60′ air may pass through the bypass 54.

FIG. 7 shows a damper 60 provided with a handle 64 which can be moved within a slot 66 in order to rotate the damper 60 about an axis 68 in order to open and close the damper 60.

1. An odour control unit comprising an input conduit, an output conduit, a means for generating ultra-violet light which in turn can generate ozone and a means for measuring the concentration of ozone present in the output conduit, wherein the rate of ozone generation can be varied depending on the concentration of ozone in the output conduit.

2. The unit according to claim 1 wherein the rate of ozone generation is varied to prevent the concentration of ozone in the output conduit from exceeding a preset value.

3. The unit according to claim 1 further comprising inner surfaces which are at least partially coated with reflective material.

4. The unit according to claim 3 wherein at least some of the inner surfaces are corrugated.

5. The unit according to claim 1 further comprising inner surfaces which are at least partially coated with material which, when ultra-violet light of wavelength approximately 254 μm is incident upon it, enters a superhydrophilic state and acts as a catalyst for the oxidisation of certain compounds contained within the odorous fumes.

6. The unit according to claim 5 wherein the superhydrophilic material is titanium dioxide in the anatase phase.

7. The unit according to claim 1 wherein the means for generating ultra-violet light is one or more pellet amalgam lamp.

8. The unit according to claim 7 wherein each lamp further comprises a shield.

9. The unit according to claim 1 further comprising a filter at the input and output conduits.

10. The unit according to claim 9 wherein the filter is substantially formed of a mesh.

11. The unit according to claim 9 wherein the filter is substantially a perforated sheet.

12. The unit according to claim 1 which is constructed as a removable cartridge.

13. A unit comprising an exhaust air treatment unit and a bypass, wherein the flow of air passing through the exhaust air treatment unit may be temporarily diverted through the bypass.

14. A unit comprising an exhaust air treatment unit and a bypass, wherein the exhaust air treatment unit is an odour control unit according to claim 1, and wherein the flow of air passing through the exhaust air treatment unit may be temporarily diverted through the bypass.

15. The unit according to claim 13 wherein the flow of air through the unit is controlled by at least one damper and at least one closing plate which determine whether the air flows through the exhaust air treatment unit or the bypass or both.

16. The unit according to claim 13 wherein the bypass is constructed integrally with the exhaust air treatment unit.

17. The unit according to claim 13 wherein the bypass is constructed separate from the exhaust air treatment unit.

18. The unit according to claim 13 wherein the bypass is a second exhaust air treatment unit.
